(19) United States
(12) Patent Application Publication
(10) Pub. No.: US 2011/0301539 A1
(43) Pub. Date: Dec. 8, 2011

(54) Fill and purge system for a drug reservoir

(76) Inventor: Matthew J.A. Rickard, Tustin, CA (US)

(21) Appl. No.: 12/795,776

(22) Filed: Jun. 8, 2010

(51) Int. Cl. A61M 5/31; A61M 1/00; A61M 39/22

(52) U.S. Cl. 604/151; 604/246; 604/247; 604/207

(57) ABSTRACT

A fill and purge system for a drug reservoir is disclosed. The fill and purge system comprises a positive pressure source, a negative pressure source, a first valve, a second valve, and a delivery channel. The positive pressure source is fluidly connected to the first valve. The negative pressure source is fluidly connected to the second valve. The first and second valves are fluidly connected to the delivery channel. A method of filling and purging a drug reservoir is also disclosed.

### FIELD OF THE INVENTION

The present disclosure relates generally to a system for filling and purging a drug reservoir.

### BACKGROUND

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

Age related macular degeneration (ARMD) is the leading cause of blindness in the elderly. ARMD attacks the center of vision and blurs it, making reading, driving, and other detailed tasks difficult or impossible. About 200,000 new cases of ARMD occur each year in the United States alone. Current estimates reveal that approximately forty percent of the population over age 75, and approximately twenty percent of the population over age 60, suffer from some degree of macular degeneration. “Wet” ARMD is the type of ARMD that most often causes blindness. In wet ARMD, newly formed choroidal blood vessels (choroidal neovascularization (CNV)) leak fluid and cause progressive damage to the retina.

In connection with CNV in ARMD, there are three main methods of treatment that are currently being developed: (a) photocoagulation, (b) the use of angiogenesis inhibitors, and (c) photodynamic therapy. Photocoagulation is the most common treatment modality for CNV. However, photocoagulation can be harmful to the retina and is impractical when the CNV is near the fovea. Furthermore, over time, photocoagulation often results in recurrent CNV. Oral or parenteral (non-ocular) administration of anti-angiogenic compounds is also being tested as a systemic treatment for ARMD. However, due to drug-specific metabolic restrictions, systemic administration usually provides sub-therapeutic drug levels to the eye. Therefore, to achieve effective intraocular drug concentrations, either an unacceptably high dose or repetitive conventional doses are required. However, patients often have compliance issues in that the medication must be applied 4 to 5 times a day to achieve therapeutic drug levels. Periocular injections of these compounds often result in the drug being quickly washed out and depleted from the eye, via periocular vasculature and soft tissue, into the general circulation. Further, repetitive intraocular injections may result in severe, often blinding, complications such as retinal detachment and endophthalmitis. Photodynamic therapy is a new technology for which the long-term efficacy is still largely unknown.

To prevent complications related to the above-described treatments and to provide better ocular treatment, researchers have suggested various implants aimed at localizing delivery of anti-angiogenic compounds to the eye. For example, one such implant is configured as a non-biodegradable polymeric implant with a pharmaceutically active agent disposed therein. The pharmaceutically active agent diffuses through the polymer body of the implant into the target tissue. The pharmaceutically active agent may include drugs for the treatment of macular degeneration and diabetic retinopathy. The implant is placed substantially within the tear fluid upon the outer surface of the eye over an avascular region, and may be anchored in the conjunctiva or sclera; episclerally or intrasclerally over an avascular region; substantially within the suprachoroidial space over an avascular region such as the pars plana or a surgically induced avascular region; or in direct communication with the vitreous.

Another exemplary implant is a polymer implant for placement under the conjunctiva of the eye. The implant may be used to deliver neovascular inhibitors for the treatment of ARMD and drugs for the treatment of retinopathies, and retinitis. The pharmaceutically active agent diffuses through the polymer body of the implant.

Yet another non-bioerodable polymer implant for delivery of certain drugs includes angiostatic steroids and drugs such as cyclosporine for the treatment of uveitis. Once again, the pharmaceutically active agent diffuses through the polymer body of the implant, but after a predetermined time, the implant is absorbed by the body. With this type of delivery configuration, periodic delivery of new implants is required for continued treatment.

All of the above-described implants require careful design and manufacture to permit controlled diffusion of the pharmaceutically active agent through a polymer body (i.e., matrix devices) or polymer membrane (i.e., reservoir devices) to the desired site of therapy. Drug release from these devices depends on the porosity and diffusion characteristics of the matrix or membrane, respectively. These parameters must be tailored for each drug moiety to be used with these devices.

An additional issue with those drug delivery devices that include a drug reservoir includes filling and purging operations. More specifically, because the pharmaceutically active agents disposed in the reservoir have predetermined shelf-lives, there is a need to periodically purge and then refill the reservoir. Currently, this process requires two channels (i.e., cannulas/needles); a first channel that is directly connected to an aspiration line and a second channel that is attached to a pressurized feed line. However, using two separate channels decreases the life of the reservoir, as repeated needle insertions creates a likelihood of a permanent tear. Moreover, a two channel arrangement also increases the likelihood of complication in that the surgeon needs to make two accurate placements of the needles into the reservoir. Thus, there exists a need for a more simplified manner to fill and purge reservoir delivery devices.

### BRIEF SUMMARY

A fill and purge system for a drug reservoir is disclosed. The fill and purge system comprises a positive pressure source, a negative pressure source, a first valve, a second valve, and a delivery channel. The positive pressure source is fluidly connected to the first valve. The negative pressure source is fluidly connected to the second valve. The first and second valves are fluidly connected to the delivery channel. A method of filling and purging a drug reservoir is also disclosed.

### BRIEF DESCRIPTION OF THE DRAWINGS

Embodiments of the present disclosure will now by described by way of example in greater detail with reference to the attached figures, in which:

FIG. 1 illustrates a schematic drawing of a single channel filling and purging system;

FIG. 2 is a schematic drawing of a surgical handpiece;

FIG. 3 is a schematic drawing of an exemplary connector device; and

FIG. 4 is a schematic drawing of an alternative pathway arrangement within a surgical handpiece.

### DETAILED DESCRIPTION

Referring now to the discussion that follows and also to the accompanying drawings, illustrative approaches to the disclosed devices and methods are shown in detail. Although the drawing figures represent some possible approaches, the drawing figures are not necessarily to scale and certain features may be exaggerated, removed, or partially sectioned to better illustrate and explain the present disclosure. Further the descriptions set forth herein are not intended to be exhaustive or otherwise limit or restrict the claims to the precise forms and configurations shown in the drawings and disclosed in the following detailed description.

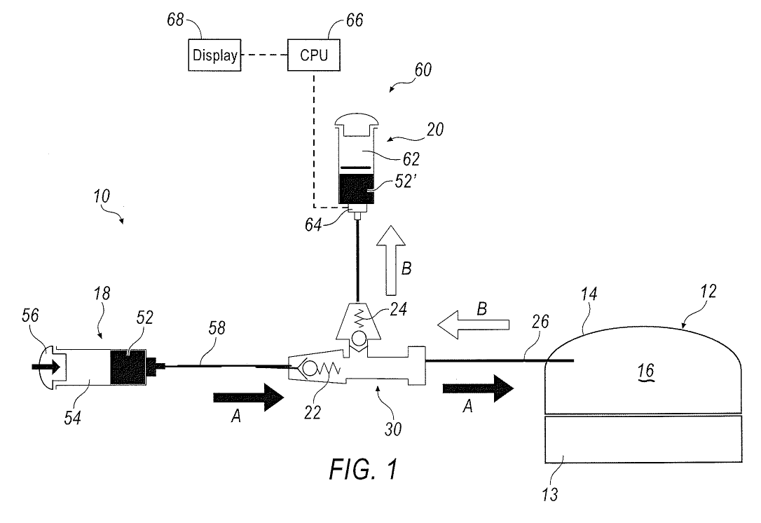

FIG. 1 is a schematic illustration of a fill and purge system 10 for a drug reservoir, including an implantable reservoir 12, such as an ocular implant. The reservoir 12 comprises a base member 13 and a flexible diaphragm 14 that defines a chamber 16 into which a pharmaceutically active agent/drug may be selectively disposed. In one arrangement, reservoir 12 is configured to be implanted into the human eye using any suitable method.

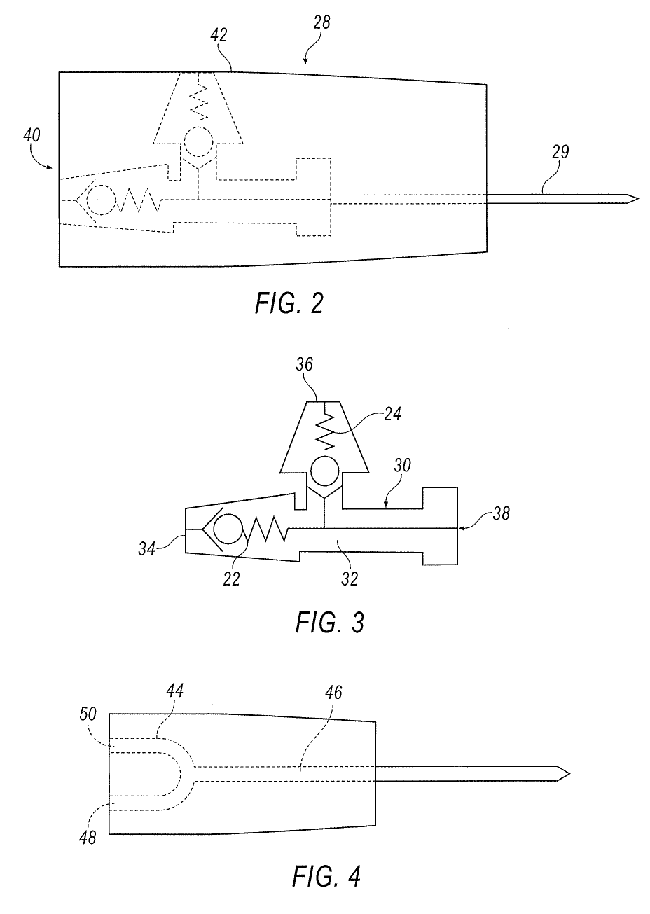

Fill and purge system 10 comprises a positive pressure source 18, a negative pressure source 20 (positive and negative pressure being defined relative to the pressure of chamber 16), first and second check valves 22, 24 and a delivery channel 26. Positive pressure source 18 is fluidly connected to first check valve 22. Negative pressure source 20 is fluidly connected to second check valve 24. In one exemplary arrangement, positive and negative pressure sources 18, 20 are integrated into a surgical console (not shown). First and second check valves 22, 24 are fluidly connected to delivery channel 26. A distal end of delivery channel 26 is configured for selective insertion through flexible diaphragm 14 of reservoir 12. Delivery channel 26 may be configured as a single use surgical handpiece 28 having a needle 29 fixedly secured thereto (shown in FIG. 2, for example).

In one exemplary arrangement, first and second check valves 22, 24 may be disposed in a unitary connector element 30. Examples of connector element 30 include a “Y” shaped connector element or a “T” shaped connector element (as shown in FIG. 3). Connector element 30 is configured with a body member 32 and first, second and third ports 34, 36, 38. First port 34 is configured with first check valve 22. Second port 36 is configured with second check valve 24. Third port 38 is configured to fluidly connect with delivery channel 26. In one exemplary arrangement connector 30 may be positioned within handpiece 28. In an alternative arrangement, connector 30 may be separate from handpiece 28 and a fluid conduit (not shown) may be positioned between third port 38 and handpiece 28 to fluidly connect third port 38 to handpiece 28.

In another alternative arrangement, stand-alone first and second check valves 22 and 24 may be individually connected to handpiece 28. For example, first check valve 22 may be secured to a proximal end 40 of handpiece 28. In one embodiment, first check valve 22 may be fixedly secured directly to handpiece 28. Alternatively, first check valve 22 may be secured to handpiece 28 through suitable disposable tubing. Similarly, second check valve 24 may also be secured to handpiece 28. In one embodiment, second check valve 24 is secured to a side wall 42 of handpiece 28. Second check valve 24 may also be secured to handpiece 28 through suitable disposable tubing.

Referring to FIG. 4, in yet another alternative arrangement, the interior of handpiece 28 may be configured with a two-channel configured pathway 44 that opens into a single pathway 46 that connects to needle 29. Ends 48, 50 of two-channel configured pathway 44 connect to first and second check valves 22, 24, in any suitable manner. In one exemplary arrangement, as shown in FIG. 4, two-channel configured pathway 44 has a generally U-shaped configuration. Other suitable configurations, such as V-shaped, are also contemplated.

In one exemplary arrangement, shown in FIG. 1, positive and negative pressure sources 18 and 20 are configured as syringes. However, it is understood that any suitable positive and negative pressure sources may be employed within system 10 without departing from the disclosure. For example, in one exemplary arrangement, syringes may be replaced with pumps (not shown).

Turning back to FIG. 1, operation of fill and purge system 10 will now be described. Once the reservoir 12 is positioned within the eye, to fill reservoir 12, a needle is used to pierce flexible diaphragm 14 that defines chamber 16. In one exemplary arrangement, needle 29 of a surgical handpiece 28 may be used. Needle 29 is configured with an opening that is in communication with delivery channel 26. A desired drug source 52 is associated with positive pressure source 18. In one exemplary arrangement, drug 52 is disposed within drug chamber 54 of a syringe. A plunger 56, or other suitable activation device, is activated to expel drug 52 from drug chamber 54 through a conduit 58 that connects positive pressure source 18 to delivery channel 26. First check valve 22 is positioned between positive pressure source 18 and delivery channel 26. First check valve 22 is a one-way valve that permits drug 52 to only flow in a first direction, i.e. toward reservoir 12. First direction is represented by arrows A.

To remove drug 52 from reservoir 12 as part of a purging operation, negative pressure source 20 creates a lower pressure (i.e., partial vacuum relative to the drug pressure) downstream of second check valve 20. In one exemplary arrangement, negative pressure source 20 creates the lower pressure by retracting a plunger 60. The retraction draws purged drug 52′ in a second direction from reservoir 12, through second check valve 24 and into a chamber 62. Second direction is represented by arrows B. Second check valve 24 is also configured as a one-way valve so as to only permit drug 54′ to flow in second direction, i.e., toward chamber 62.

Because system 10 includes a single delivery channel 26, the number of needles previously required to perform the filling and purging operation is reduced. Indeed, complications of the filling and purging procedure are reduced as the surgeon need only make a single insertion into reservoir 12, rather than placing two separate devices. Further, the useful life of the reservoir 12 may also be increased as only one tear is created through diaphragm 14 per operation, rather than multiple operations.

The filling and purging steps may be repeated such that chamber 16 will be continually purged. More specifically, new drug 54 will flow into chamber 16 and then be removed. By continuously repeating the filling and purging process, the older quantity of drug 52, as well as contaminants within chamber 16, will be removed therefrom. Moreover, with each successive iteration of the filling and purging process, the concentration of the new drug 52 in chamber 16 will continue to increase. The process is asymptotic in nature; the most dramatic improvements of new drug 52 concentrations occur during the early stages of the process and diminishing returns are realized each time the purge process is repeated.

In one embodiment, negative pressure source 20 is operatively connected to a drug chamber 62 that is selectively removable from system 10 such that the purged drug 52′ contained therein may be removed and evaluated. More specifically, purged drug 52′ may be tested at periodic intervals in the filling and purging process to ascertain when a predetermined concentration of purged drug 52′ has been achieved. In another configuration, a sensor 64 may be operatively connected to negative pressure source 20 that evaluates purged drug 52′ during the filling and purging process. Sensor 64 sends a signal to a central processing unit (CPU) 66 and provides a notification to the user if purged drug 52′ has a predetermined concentration. In one arrangement, when purged drug 52′ reaches the predetermined concentration, CPU 66 sends a signal to a display 68, such as may be found on a surgical console. An audible signal may also be generated. In one particular arrangement, the testing/verification step may be done while needle 29 is still positioned within the chamber 16, thereby reducing multiple insertions in the eye.

It will be appreciated that the devices and methods described herein have broad applications. The foregoing embodiments were chosen and described in order to illustrate principles of the methods and apparatuses as well as some practical applications. The preceding description enables others skilled in the art to utilize methods and apparatuses in various embodiments and with various modifications as are suited to the particular use contemplated. In accordance with the provisions of the patent statutes, the principles and modes of operation of this invention have been explained and illustrated in exemplary embodiments.

It is intended that the scope of the present methods and apparatuses be defined by the following claims. However, it must be understood that this invention may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future examples. Furthermore, all terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the invention and that the method and apparatus within the scope of these claims and their equivalents be covered thereby. In sum, it should be understood that the invention is capable of modification and variation and is limited only by the following claims.

What is claimed is:

1. A fill and purge system for a drug reservoir, comprising:
a positive pressure source;
a negative pressure source;
a first valve;
a second valve; and
a delivery channel;
wherein the positive pressure source is fluidly connected to the first valve and the negative pressure source is fluidly connected to the second valve; and
wherein the first and second valves are fluidly connected to the delivery channel.

2. The fill and purge system of claim 1, wherein the delivery channel is a surgical handpiece.

3. The fill and purge system of claim 2, wherein the surgical handpiece further comprises a needle fixedly secured thereto, the needle being configured for insertion into the drug reservoir.

4. The fill and purge system of claim 1, further comprising a connector element, wherein the first and second valves are connected to the connector element.

5. The fill and purge system of claim 4, wherein the connector element further comprises first, second and third ports;
wherein the first valve is operatively connected to the first port;
wherein the second valve is operatively connected to the second port; and
wherein the third port is configured to operatively connect to the delivery channel.

6. The fill and purge system of claim 5, wherein the connector element is disposed within a surgical handpiece and the delivery channel includes a proximal end that is position within the handpiece and connected to the third port of the connector.

7. The fill and purge system of claim 6, wherein the first and second valves are each connected to a portion of the handpiece.

8. The fill and purge system of claim 5, wherein at least one of the first and second valves are integrally connected to the connector element.

9. The fill and purge system of claim 5, wherein at least one of the first and second valves are operatively connected to the connector element by tubing.

10. The fill and purge system of claim 5, wherein the third port of the connector element is operatively connected to the delivery channel by tubing.

11. The fill and purge system of claim 1, wherein the first and second valves are configured as one-way check valves.

12. The fill and purge system of claim 11, wherein the first valve is configured to permit a flow of material in a first direction, away from the positive pressure source.

13. The fill and purge system of claim 12, wherein the second valve is configured to permit a flow of material in a second direction, toward the negative pressure source.

14. The fill and purge system of claim 1, wherein at least one of the positive and negative pressure sources is a pump.

15. The fill and purge system of claim 1, wherein at least one of the positive and negative pressure sources is a syringe.

16. The fill and purge system of claim 1, further comprising a drug chamber operatively connected to each of the positive and negative pressure sources, wherein the drug chamber operatively connected to the negative chamber receives material aspirated from the drug reservoir, and wherein the drug chamber operatively connected to the positive pressure sources receives material to be introduced into the drug reservoir.

17. The fill and purge system of claim 16, wherein the drug chamber operatively connected to the negative pressure source is selectively detachable from the system.

18. The fill and purge system of claim 16, further comprising a sensor that is configured to detect a predetermined concentration of the material being aspirated into the drug chamber.

19. A method of filling and purging a drug reservoir, comprising:
providing positive and negative pressure sources that are operatively connected to a first opening of a delivery channel, wherein the positive pressure source is fluidly connected to a first check valve and the negative pressure source is fluidly connected to a second check valve;
providing a source of material to be disposed within the drug reservoir, wherein the source of the material is fluidly connected to the positive pressure source and is positioned downstream of the first check valve;
positioning the delivery channel so as to have a second opening thereof in communication with the drug reservoir;
activating the positive pressure source to direct the material in a first direction, through the first check valve to the delivery channel such that the material is deposited into the drug reservoir; and
activating the negative pressure source that is operatively connected to a storage chamber to direct material disposed within the drug reservoir through the delivery channel in a second direction, through the second check valve and into the storage chamber.

20. The method of claim 19, further comprising repeating the steps of activating the positive pressure source and activating the negative pressure source a predetermined number of times until the concentration of the material reaches a predetermined level.

21. The method of claim 19, further comprising selectively detaching the storage chamber from the negative pressure source after the step of activating the negative pressure source and evaluating the concentration of the material disposed therein.

22. The method of claim 19, further comprising activating a sensor to evaluate the concentration of the material disposed within the storage chamber.
